(19) United States
(12) Patent Application Publication
(10) Pub. No.: US 2006/0282169 A1
(43) Pub. Date: Dec. 14, 2006

(54) System and method for upper extremity joint arthroplasty

(76) Inventors: Jeffrey C. Felt, Greenwood, MN (US); Mark A. Rydell, Golden Valley, MN (US); Michael B. Purnell, Modesto, CA (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(21) Appl. No.: 11/311,894

(22) Filed: Dec. 19, 2005

Related U.S. Application Data
(60) Provisional application No. 60/637,136, filed on Dec. 17, 2004.

(51) Int. Cl. A61F 2/38

(52) U.S. Cl. 623/20.11; 623/23.44

(57) ABSTRACT

The invention can provide an implant for an upper extremity joint arthroplasty having a first polymeric portion adapted to be retained within a bone of the upper extremity and a second polymeric portion having an articulating surface adapted to articulate against an opposing bone. The invention can also provide methods of making and using such an implant.

### RELATED APPLICATIONS

This application claims priority to U.S. Provisional Patent Application Ser. No. 60/637,136, titled Radial Head and Distal Ulna Prosthesis, filed Dec. 17, 2004, the contents of which are hereby incorporated by reference.

### TECHNICAL FIELD

In one aspect, this invention relates to materials for implantation and use within the body. In another aspect, this invention further relates to the field of orthopedic implants and prosthesis, and more particularly, for implantable materials for use in joint arthroplasty.

### BACKGROUND OF THE INVENTION

Radioulnar joints are synovial joints of the upper extremity. The proximal joint lies between the head of radius and the radial notch of the ulna, while the distal joint is separated from the wrist by an articular disc that extends from base of ulnar styloid process to the radius. The radial head articulates at the elbow with the olecranon process of the ulna and rotates in the intercondylar fossa between the medial and lateral condyle of the distal humorous. Rotation of the radial head allows supination and pronation of the forearm and hand. These motions allow for the positioning of the hand for proper fine motor movements. Loss of radial head rotation or significant pain from motion will significantly impact fine motor skills of a patient. The distal ulna is the end of the ulna farthest from the body that forms a joint between the distal ulna, the distal radius, and the carpals of the wrist.

There are several conditions in which radio-ulnar joint motion is impaired or lost completely. These include rheumatoid or other forms of inflammatory arthritis, aseptic necrosis of the radial head, trauma, posttraumatic osteoarthritis and metastatic or primary bone tumors. Similar conditions can cause damage to the distal ulna.

The current treatment for loss of radial head function is replacement of the radial head with a prosthesis consisting of a hard material having an extremely high modulus of elasticity. Generally, these implants consist of a rod extending into the medullary shaft of the radius with a proximal rounded cylindrical head to duplicate the normal anatomy of the radial head. Unfortunately, the significant hardness and modulus mismatch between the implant and the bone that it must articulate with leads to milling and erosions in the adjacent bone with resultant pain, swelling and reduced function. Further, it can ultimately lead to failure of the implant. The treatment and failure modes for the distal ulna are analogous to those seen with the radial head.

### SUMMARY OF THE INVENTION

In some embodiments, the invention provides an implant for an upper extremity joint arthroplasty, the implant comprising a first polymeric portion adapted to be retained within a bone of the upper extremity and a second polymeric portion having an articulating surface adapted to articulate against an opposing bone. In some embodiments, the bone of the upper extremity is a radius and the opposing bone is selected from the group of an ulna, a humorous, and combinations thereof (i.e., the elbow). In other embodiments, the bone of the upper extremity is an ulna and the opposing bone is selected from the group of a radius, a carpal, and combinations thereof (i.e., the wrist).

The implant can comprise one or more biomaterials selected from the group of polyurethane, polyethylene, poly(ether ether ketone), polyurethane functionalized polyethylene mixes, polyurethane siloxane mixtures, polystyrene, polyamides, silicones, acrylics and combinations thereof, as well as other biomaterials discussed herein.

In some embodiments, the first polymeric portion comprises a shaft portion and the second polymeric portion comprises a head portion. In such embodiments, the head portion can comprise a relatively harder biomaterial than the shaft portion. Further, both the head portion and the shaft portion can comprise a material having a softer modulus than natural bone.

In some embodiments, the invention described herein includes a system comprising an implant with a compressive modulus softer than bone for an upper extremity joint (e.g., synovial joint) arthroplasty. In some embodiments, the implant comprises a highly stress fatigue and wear resistant biomaterial. For example, the compressive modules can be low enough to allow the material be inserted into a shaft defined within the radius or ulna (e.g., the medullary shaft of the radius). In some embodiments, the compressive modulus of the material can be low enough to allow the material to bend while it is inserted into such a shaft. Further, a high stress fatigue material wear resistant material can relate to the hardness and/or compression modulus of the material. For example, in embodiments of the implant that have a head portion, the head portion can comprise a material having a hardness of about 55 Shore D to about 75 Shore D (e.g., about 65 Shore D) and a compression modulus of about 10,000 pounds per square inch (psi) to about 13,000 psi (e.g., about 11,500 psi). In embodiments having a shaft portion, the shaft portion can comprise a material having a hardness of about 80 Shore A to about 90 Shore A (e.g. about 85 Shore A), and a compressive modulus of about 1,500 psi to about 4,500 psi (e.g., about 3,000 psi).

The invention also includes a method of placing an implant in an upper extremity joint, the method comprising providing an implant comprising a first polymeric portion and a second polymeric portion having an articulating surface, inserting the first polymeric portion within a bone of the upper extremity, and placing the articulating surface against an opposing bone.

Some embodiments of the system can also include one or more devices in the form of a kit that can be used to provide or perform some or all of the steps of preparing the joint to receive an implant, determining an appropriate implant size for a particular joint, determining an appropriate implant thickness and/or angle, inserting the implant into the joint, and/or securing the implant to a desired extent. One or more of the various components and devices, including optionally one or more implants themselves, can be provided or packaged separately or in varying desired combinations and subcombinations to provide a kit of this invention.

### BRIEF DESCRIPTION OF THE DRAWING

In the Drawing:

FIG. 1(a) shows a perspective view of an implant in accordance with an embodiment of the invention.

FIG. 1(b) shows a side plan view of an implant in accordance with an embodiment of the invention.

FIG. 1(c) shows a sectional view of an implant in accordance with an embodiment of the invention.

FIG. 2(a) shows a perspective view of an implant in accordance with an embodiment of the invention.

FIG. 2(b) shows a side plan view of an implant in accordance with an embodiment of the invention.

FIG. 2(c) shows a sectional view of an implant in accordance with an embodiment of the invention.

FIG. 3 shows a front plan skeletal view of a left human arm with the palm up having implants in accordance with an embodiment of the invention.

### DETAILED DESCRIPTION

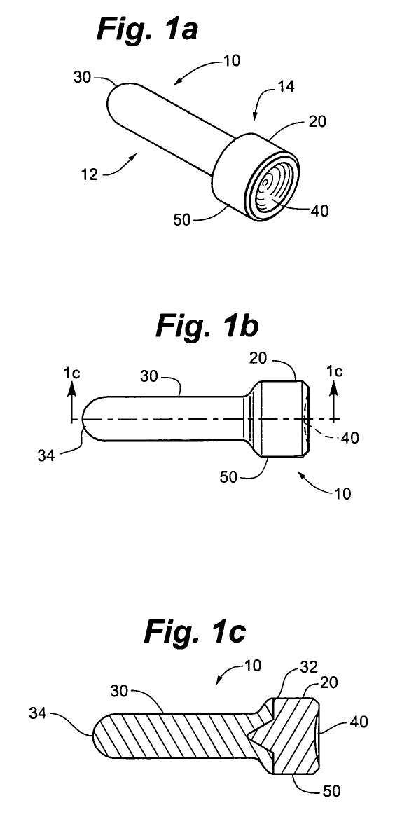

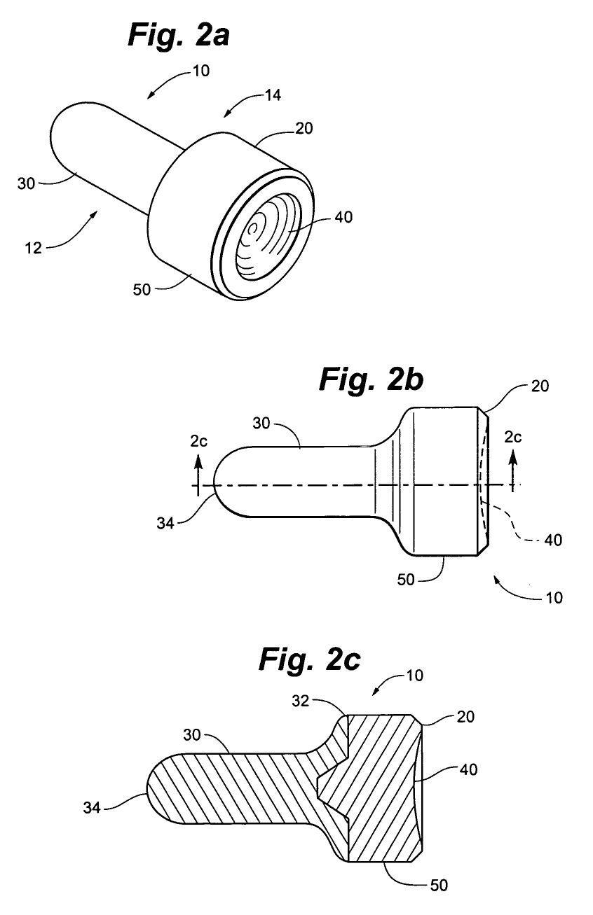

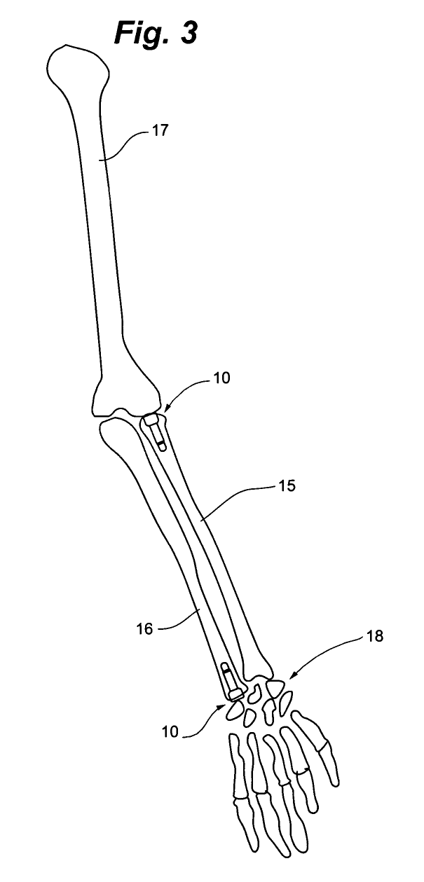

A preferred embodiment of the method and implant of this invention will be further described with reference to the Drawing, in which FIGS. 1(a)-2(c) show various views of an implant 10 in accordance with embodiments of the invention. In some embodiments, as in FIGS. 1(a) and 2(a), the invention provides an implant for an upper extremity joint arthroplasty, the implant comprising a first polymeric portion 12 adapted to be retained within a bone of the upper extremity and a second polymeric portion 14 having an articulating surface adapted to articulate against an opposing bone. As shown in FIG. 3, in some embodiments the bone of the upper extremity is a radius 15 and the opposing bone is selected from the group of an ulna 16, a humorous 17, and combinations thereof (i.e., the elbow). In other embodiments, the bone of the upper extremity is an ulna 16 and the opposing bone is selected from the group of a radius 15, a carpal 18, and combinations thereof (i.e., the wrist).

Implant 10 can comprise a head portion 20 and a shaft portion 30. The implant 10 can comprise a plurality of biomaterials, and preferably includes a polymeric material. In turn, the implant can be prepared from a plurality of components, of the same or different materials. For example, head portion 20 and shaft portion 30 can comprise a single component or can be two or more components functionally coupled together about a junction 32, as shown in FIGS. 1(c) and 2(c). In some embodiments, the junction 32 comprises a Morris taper.

Such an implant 10 can be relatively soft, and in some embodiments, both the head portion 20 and the shaft portion 30 comprise a material having a softer modulus than natural bone. In some embodiments, shaft portion 30 comprises a softer durometer elastomeric material than head portion 20 to facilitate insertion into the receiving bone (e.g., medullary shaft of the radius). For example, head portion 20 can comprise a polyurethane and the shaft portion 30 can comprise a relatively softer polyurethane. In such embodiments, the two different polyurethanes can be co-polymerized to form a continuous polyurethane material across junction 32 of the head portion and the shaft portion of the implant.

In some embodiments, head portion 20 comprises an articulating surface 40 useful for providing a surface for articulation in an upper extremity synovial joint. As best shown in FIGS. 1(a), 1(c), 2(a) and 2(c), articulating surface can comprise a generally concave surface useful for approximating the shape of natural bone.

Head portion 20 can comprise any suitable shape. For example, head portion 20 can comprise a cylindrical shape having radiused edges. Further, head portion 20 can comprise a body region 50 having a depth useful for filing space within a joint. In some embodiments, the head portion 20 comprises a cylinder shape with radiused edges to mimic the structure and function of the original radial head or distal ulna.

Head portion 20 can comprise any suitable diameter or length. In the embodiments of FIGS. 1(a)-1(c), body region 50 is relatively less prominent and can be particularly useful in a distal ulna joint. By contrast, the body region 50 of FIGS. 2(a)-(c) is relatively more prominent and can be particularly useful for in a radial head joint. For example, in embodiments adapted for the radial head, the head portion diameter can be about 15 millimeters (mm) to about 25 mm (e.g., about 20 mm) and the head portion length can be about 8 mm to about 16 mm (e.g., about 12 mm). In embodiments adapted for the distal ulna, for example, the head portion diameter can be about 10 mm to about 20 mm (e.g., about 15 mm) and the head portion length can be about 10 mm to about 20 mm (e.g., about 15 mm). It should be noted that the numbers given above are merely representative, and that other sizes or shapes can be provided without departing from the scope of the invention.

Shaft portion 30 can be adapted to be received within a bone of an upper extremity, such as a radius or ulna. In addition, the shaft portion 30 can comprise any suitable shape, including a cylindrical shape with a rounded bullet shaped end 34, as shown in FIGS. 1(b) and (c) and 2(b) and (c). Such a shape is adapted to ease insertion of the shaft portion 30 into the bone of the upper extremity. In embodiments wherein the head portion 20 and the shaft portion 30 each comprise a cylindrical shape, the diameter of the head portion 20 can be greater than the diameter of the shaft portion 30. In an alternate embodiment, the shaft portion 30 of the implant 10 has a pentagonal or hexagonal configuration to improve rotational stability of the implant 10 while avoiding stress concentrations that can occur with a triangular or square shaft profile.

Further, shaft portion 30 can comprise any suitable thickness and length to provide for fixation and stability of the prosthesis 10. In the embodiments of FIGS. 1(a)-1(c), shaft portion 30 has a relatively elongated shape that can be particularly useful in a distal ulna joint. By contrast, the shaft portion 30 of FIGS. 2(a)-(c) can be particularly useful for a radial head joint. For example, in embodiments adapted for the radial head, the shaft portion diameter can be about 8 mm to about 15 mm (e.g., about 11 mm) and the shaft portion length can be about 35 mm to about 55 mm (e.g., about 43 mm). In embodiments adapted for the distal ulna, for example, the shaft portion diameter can be about 5 mm to about 15 mm (e.g., about 9 mm) and the shaft portion length can be about 20 mm to about 35 mm (e.g., about 25 mm). Again, it should be noted that the numbers given above are merely representative, and that other sizes or shapes can be provided without departing from the scope of the invention.

Some embodiments of the system can also include one or more devices in the form of a kit, that can be used to provide or perform some or all of the steps of preparing the joint to receive an implant, determining an appropriate implant size for a particular joint, determining an appropriate implant thickness and/or angle, inserting the implant into the joint, and/or securing the implant to a desired extent. One or more of the various components and devices, including optionally one or more implants themselves, can be provided or packaged separately or in varying desired combinations and subcombinations to provide a kit of this invention.

In some embodiments, several implants are included in the kit. For example, implants can be provided in various head portion 20 and shaft portion 30 diameters and lengths to accommodate structural variations of the joint dimensions. Further, in some embodiments, at least one trial implant can be provided for confirming the proper implant diameter and length before the implant is placed.

In some embodiments, at least one reamer can be provided for removing bone and natural tissues in the joint so that the implant can be placed. Such reamers can be powered by a surgical drill.

In some embodiments, at least one impactor useful for placing the implant within a joint can be provided. In some embodiments, impactor is designed to hold an implant at one end, and to be struck by a mallet at the opposite end to place an implant within a joint. In some embodiments, impactors of several diameters are provided in order to hold implants of different sizes.

In some embodiments, at least one bone smoother can be provided for smoothing and removal of marginal osteophytes from the remaining natural joint surfaces that would interfere with the motion of the implant.

The tools described above can be constructed of any suitable material. For example, the tools can be constructed of stainless steel, ceramic, and/or polymeric materials. Embodiments constructed at least partially of stainless steel can be relatively more suitable for providing a reusable tool, and embodiments constructed at least partially of a polymer can be relatively more suitable for providing a disposable tool. Further, all of the tools above can be shaped to provide an ergonomic fit for the user. Some embodiments provide a universal tool that is configured to provide an ergonomic fit for both left and right hands and/or can be used both the right and left upper extremities.

Methods in accordance with embodiments of the invention include placing any of the various implants discussed herein. In some embodiments, access to the joint is provided such as by incision and/or dislocation of the joint. In one exemplary method in accordance with the present invention, an incision is made at a location proximate the elbow or the wrist and the joint is dis-articulated and the end of the radius or ulna exposed. A reamer can be used to create a tapered countersunk area for the implant to reside in. The implant can be, for example, hammered into this hole in a press fit relationship. The joint is placed back into position and the tissue suture is closed.

In some embodiments, the invention includes a method of placing an implant in an upper extremity joint, the method comprising providing an implant comprising a first polymeric portion and a second polymeric portion having an articulating surface, inserting the first polymeric portion within a bone of the upper extremity, and placing the articulating surface against an opposing bone.

Fixation methods beyond the retention of the shaft portion 30 within a receiving bone can also be provided and can include the use of biologic glues to glue the implant to the underlying surface, using various anchors to the underlying structure and fixing the implant to that surface or using mold retainers and/or screws, staples, sutures or pins. In an alternative embodiment, anchors in the underlying structure can be used for fixing the implant to that surface and the implant can be adapted to permit or encourage the growth of tissue thereon, and/or by encapsulation, to secure anchoring.

By the selection and use of suitable biomaterials, and other features as described herein, the present invention provides an optimal combination of benefits, as compared to methods previously described. Such benefits include those that arise in the course of preparation and storage (e.g., sterility, storage stability), those that arise in the surgical field itself (e.g., ease of use, adaptability, predictability), and those that arise in the course of long term use within the body (e.g., biocompatibility, moisture cure characteristics, tissue congruity and conformability, retention, wear characteristics, and physical-mechanical properties).

In one preferred embodiment, the method and implant involve the preparation and use of polymeric components that can be formed outside the body, for insertion and placement into the body, and that can then be further formed within the joint site in order to enhance conformance. The optional ability to finally form one or more components in situ provides various additional benefits, such as increased control over the overall size and shape of the final prosthesis, improved shape and compliance of the surface apposing natural bone, and finally, improved shape and compliance of the opposite, articulating surface.

The implantable material for use in resurfacing a joint can be formed ex vivo as an injectable material that sets up to the molded shape. Various methods for changing the state of a composition from liquid to a suitable solid include a suitable combination of heating (or cooling) time, and chemical reactivity. A chemical reaction can be exothermic or endothermic, and can be initiated in any suitable manner, e.g., activated by light and/or heat, or chemically catalyzed. Examples of such implants include flowable polymers of two or more components, light activated polymers, and polymers cured either by the use of catalysts or by heat, including body heat, or any suitable combination thereof. As a further embodiment, the material can be synthesized ex vivo and then machined to fit, using imaging and/or to a predetermined geometry and size of the implant.

Biomaterials

Both the preformed component(s) and flowable biomaterial, if used, can be prepared from any suitable material, including metals, ceramics, and/or polymers. Generally, a material is suitable if it has appropriate biostability, biodurability and biocompatibility characteristics. In some embodiments, the materials include polymeric materials, having an optimal combination of such properties as biostability, biodurability, biocompatibility, physical strength and durability, and compatibility with other components (and/or biomaterials) used in the assembly of a final composite.

Examples of polymeric materials that can be suitable in some applications, either alone or in combination, include polyurethane, available from Polymer Technology Group Incorporated under the names Bionate,™ Biospan,™ and Elasthane™, available from Dow Chemical Company under the name Pellethane,™ and available from Bayer Corp. under the names Bayflex,™ Texin,™ and Desmopan;™ ABS, available from GE Plastics under the name Cycolac™, and available from Dow Chemical Company under the name Magnum;™ SAN, available from Bayer Plastics under the name Lustran;™ Acetal, available from Dupont under the name Delrin,™ and available from Ticona GmbH and/or Ticona LLC (Ticona) under the name Celcon;™ polycarbonate, available from GE Plastics under the name Lexan,™ and available from Bayer Corp. under the name Makrolon;™ polyethylene, available from Huntsman LLC, and available from Ticona under the names GUR 1020™ and GUR 1050;™ polypropylenes, available from Solvay Engineered Polymers, Inc. under the name Dexflex;™ aromatic polyesters, available from Ticona; polyetherimide (PEI), and available from GE Plastics under the name Ultem;™ polyamide-imide (PAI), available from DSM E Products under the name Torlon;™ polyphenylene sulfide, available from Chevron Phillips Chemical Company LP under the name Ryton;™ polyester, available from Dupont under the name Dacron;™ polyester thermoset, available from Ashland Specialty Chemical Company under the name Aropol;™ polyureas; hydrogels, available from Hydromer Inc.; liquid crystal polymer, available from Ticona under the name Vectra;™ polysiloxanes, available from Nusil Technologies, Inc.; polyacrylates, available from Rohm & Haas under the name Plexiglas;™ epoxies, available from Ciba Specialty Chemicals; polyimides, available from Dupont under the names Kapton,™ and Vespel;™ polysulfones, available from BP Amoco Chemicals under the name Udel,™ and available from BASF Corporation under the name Ultrason;™ PEAK/PEEK, available from Victrex under the name Victrex PEAK;™ as well as biopolymers, such as collagen or collagen-based materials, chitosan and similar polysaccharides, and combinations thereof. Of course, any of the materials suitable for use in a composite or single biomaterial implant can be structurally enhanced with fillers, fibers, meshes or other structurally enhancing means.

Optionally and preferably, the implant comprises a polyurethane, and more preferably, one that includes one or more fillers, e.g., reactive or surface-modified particles or fibers, in order to improve and/or confer desirable physical or mechanical properties. Such fillers can be incorporated (e.g., generally admixed) at any suitable time and in any suitable manner in the course of fabricating a polymeric implant of this invention. Suitable weight ranges for such fillers, for instance, are typically between about 1 and about 50 percent, based on the weight of the implant, preferably between about 10 and about 30 percent by weight. Preferred fillers provide an optimal combination of such properties as compatibility with the polymer of choice, biocompatibility, and the ability to be sterilized.

Examples of suitable additives include ultrahigh molecular weight polyethylene (e.g., in particle sizes ranging from 180 microns to 35 microns), high density polyethylene particles and fibers (e.g., in particle sizes ranging from 500 microns to 18 microns). Such additives are commercially available, including those available under the Inhance tradename, from the Inhance Group of Fluoro-Seal, Houston, Tex. A particularly preferred additive is an ultrahigh molecular weight polyethylene particle, available under the tradename Inhance UH particles. By way of example, surface modified particles of this type were added to polyurethane compositions of the type described herein, and resulting cured compositions were tested for wear resistance sing an abrasive wheel and specimen holder with applied load. Applicants found on average a 35% reduction in wear with the particle-containing compositions as compared to those without the particles.

The present invention further provides a biomaterial having an improved combination of properties for the preparation, storage, implantation and long term use of medical implants. The improved properties correspond well for the preparation and use of an implant having both weight bearing and/or articulating functions.

In turn, a preferred biomaterial of this invention provides an optimal combination of properties relating to wear resistance, congruence, and cushioning while meeting or exceeding requirements for biocompatibility, all in a manner that serves to reduce the coefficient of friction at the major motion interface.

Wear resistance can be assessed by determining parameters such as DIN abrasion and flexural stress strain fatigue resistance. A preferred implant will have sufficient wear resistance to avoid the generation of clinically significant particulate debris over the course of the implant's use.

Congruence can be assessed by determining parameters such as tensile modulus compressive modulus, and hardness, to determine the manner and extent to which the implant will conform itself to possible other components of the implant itself and/or to bone or surrounding tissue.

Cushioning can be assessed by determining such parameters as hardness, compressive modulus, and tensile modulus, to determine the elastomeric nature of the material, and in turn, its suitability for use in a weight bearing joint. More elastomeric materials will generally provide greater comfort in weight bearing applications, particularly if the other physical properties can be maintained.

Wear resistance, congruence, and/or cushioning toughness can be achieved without undue effect on other desired properties, such as abrasion, hardness, specific gravity, tear resistance, tensile strength, ultimate elongation, and biocompatibility. Moreover, Applicant has discovered that such properties can themselves be provided in varying forms, as between first and second biomaterials of a composite of the present invention.

A polymeric biomaterial of this invention can be prepared using any suitable means, including by curing the polymer ex vivo. The composition can be used in any suitable combination with other materials, including other compositions of the same or similar nature, as well as other materials such as natural or synthetic polymers, metals (e.g., titanium, nitinol, and/or combinations or alloys thereof), ceramics, and the like.

The biomaterial used in this invention preferably includes polyurethane components that are reacted ex vivo to form a polyurethane (“PU”). The formed PU, in turn, includes both hard and soft segments. The hard segments are typically comprised of stiffer oligourethane units formed from diisocyanate and chain extender, while the soft segments are typically comprised of one or more flexible polyol units. These two types of segments will generally phase separate to form hard and soft segment domains, since they tend to be incompatible with one another. Those skilled in the relevant art, given the present teaching, will appreciate the manner in which the relative amounts of the hard and soft segments in the formed polyurethane, as well as the degree of phase segregation, can have a significant impact on the final physical and mechanical properties of the polymer. Those skilled in the art will, in turn, appreciate the manner in which such polymer compositions can be manipulated to produce cured and curing polymers with desired combination of properties within the scope of this invention.

The hard segments of the polymer can be formed by a reaction between the diisocyanate or multifunctional isocyanate and chain extender. Some examples of suitable isocyanates for preparation of the hard segment of this invention include aromatic diisocyanates and their polymeric form or mixtures of isomers or combinations thereof, such as toluene diisocyanates, naphthalene diisocyanates, phenylene diisocyanates, xylylene diisocyanates, and diphenylmethane diisocyanates, and other aromatic polyisocyanates known in the art. Other examples of suitable polyisocyanates for preparation of the hard segment of this invention include aliphatic and cycloaliphatic isocyanates and their polymers or mixtures or combinations thereof, such as cyclohexane diisocyanates, cyclohexyl-bis methylene diisocyanates, isophorone diisocyanates and hexamethylene diisocyanates and other aliphatic polyisocyanates. Combinations of aromatic and aliphatic or arylakyl diisocyanates can also be used.

The isocyanate component can be provided in any suitable form, examples of which include 2,4′-diphenylmethane diisocyanate, 4,4′-diphenylmethane diisocyanate, and mixtures or combinations of these isomers, optionally together with small quantities of 2,2′-diphenylmethane diisocyanate (typical of commercially available diphenylmethane diisocyanates). Other examples include aromatic polyisocyanates and their mixtures or combinations, such as are derived from phosgenation of the condensation product of aniline and formaldehyde. It is suitable to use an isocyanate that has low volatility, such as diphenylmethane diisocyanate, rather than more volatile materials such as toluene diisocyanate. An example of a particularly suitable isocyanate component is the 4,4′-diphenylmethane diisocyanate (“MDI”). Alternatively, it can be provided in liquid form as a combination of 2,2′-, 2,4′- and 4,4′-isomers of MDI. In a preferred embodiment, the isocyanate is MDI and even more preferably 4,4′-diphenylmethane diisocyanate.

In one embodiment of the invention, the isocyanate is 4,4′-diphenylmethane diisocyanate (as available from Bayer under the tradename Mondur M), from preferably about 20 to 60 weight percent, more preferably from about 30 to 50 weight percent. The actual amount of isocyanate used should be considered in combination with other ingredients and processing parameters, particularly including the amount of chain extender (such as butanediol (BDO)) used, since the combination typically determines the hard segment component, and in turn hardness, of the corresponding cured polymer. Hardness correlates in a generally proportional fashion with the combined weights of MDI and BDO, such that compositions having between 30 and 60 total weight percent (MDI+BDO) are generally useful, with those compositions having between about 50 to about 60 total weight percent being somewhat harder. By contrast, compositions having between about 40 to about 50 total weight percent are somewhat more congruent and cushioning, though less wear resistant.

Some examples of chain extenders for preparation of the hard segment of this invention include, but are not limited, to short chain diols or triols and their mixtures or combinations thereof, such as 1,4-butane diol, 2-methyl-1,3-propane diol, 1,3-propane-diol ethylene glycol, diethylene glycol, glycerol, tri-methylpropane, cyclohexane dimethanol, triethanol amine, and methyldiethanol amine. Other examples of chain extenders for preparation of the hard segment of this invention include, but are not limited to, short chain diamines and their mixtures or combinations thereof, such as dianiline, toluene diamine, cyclohexyl diamine, and other short chain diamines known in the art.

The soft segment consists of urethane terminated polyol moieties, which are formed by a reaction between the polyisocyanate or diisocyanate or polymeric diisocyanate and polyol. Examples of suitable diisocyanates are denoted above. Some examples of polyols for preparation of the soft segment of this invention include but are not limited to polyalkylene oxide ethers derived form the condensation of alkylene oxides (e.g. ethylene oxide, propylene oxide, and blends thereof), as well as tetrahyrofuran based polytetramethylene ether glycols, polycaprolactone diols, polycarbonate diols and polyester diols and combinations thereof. In a preferred embodiment, the polyols are polytetrahydrofuran polyols (“PTHF”), also known as polytetramethylene oxide (“PTMO”) or polytetramethylene ether glycols (“PTMEG”). Even more preferably, the use of two or more of PTMO diols with different molecular weights selected from the commercially available group consisting of 250, 650,1000, 1400, 1800, 2000 and 2900.

Two or more PTMO diols of different molecular weight can be used as a blend or separately, and in an independent fashion as between the different parts of a two part system. The solidification temperature(s) of PTMO diols is generally proportional to their molecular weights. The compatibility of the PTMO diols with such chain extenders as 1,4-butanediol is generally in the reverse proportion to the molecular weight of the diol(s). Therefore the incorporation of the low molecular weight PTMO diols in a “curative” (part B) component of a two part system, and higher molecular weight PTMO diols in the prepolymer (part A) component, can provide a two-part system that can be used at relatively low temperature. In turn, good compatibility of the low molecular weight PTMO diols with such chain extenders as 1,4-butanediol permits the preparation of two part systems with higher (prepolymer to curative) volume ratio. Amine terminated polyethers and/or polycarbonate-based diols can also be used for building of the soft segment.

In one embodiment of the invention, the polyol is polytetramethyleneetherglycol 1000 (as available from E.I. du Pont de Nemours and Co. under the tradename Terathane 1000), preferably from about 0 to 40 weight percent, more preferably from about 10 to 30 weight percent, and perhaps even more preferably from about 22 to 24 weight percent, based on the total weight of the polymer. The polyol disclosed above can be used in combination with polytetramethyleneetherglycol 2000 (as available from E.I. du Pont de Nemours and Co. under the tradename Terathane 2000), preferably from about 0 to 40 weight percent, more preferably from about 10 to 30 weight percent, and perhaps even more preferably from about 17 to 18 weight percent, based on the total weight of the polymer.

In one embodiment, the biomaterial can include a chain extender. For example, the chain extender can be 1,4-butanediol (as available from Sigma Aldrich Corp.), preferably from about 1 to 20 weight percent, more preferably from 5 to 15 weight percent, to perhaps even more preferably from 12 to 13 weight percent, based on the total weight of the polymer.

The polyurethane can be chemically crosslinked, e.g., by the addition of multifunctional or branched OH-terminated crosslinking agents or chain extenders, or multifunctional isocyanates. Some examples of suitable crosslinking agents include, but are not limited to, trimethylol propane (“TMP”), glycerol, hydroxyl terminated polybutadienes, hydroxyl terminated polybutadienes (HOPB), trimer alcohols, Castor oil polyethyleneoxide (PEO), polypropyleneoxide (PPO) and PEO-PPO triols. In a preferred embodiment, HOPB is used as the crosslinking agent.

This chemical crosslinking augments the physical or “virtual” crosslinking of the polymer by hard segment domains that are in the glassy state at the temperature of the application. The optimal level of chemical cross-linking improves the compression set of the material, reduces the amount of the extractable components, and improves the biodurability of the PU. This can be particularly useful in relatively soft polyurethanes, such as those suitable for the repair of damaged cartilage. Reinforcement by virtual cross-links alone may not generate sufficient strength for in vivo performance in certain applications. Additional cross-linking from the soft segment, potentially generated by the use of higher functional polyols can be used to provide stiffer and less elastomeric materials. In this manner a balancing of hard and soft segments, and their relative contributions to overall properties can be achieved.

In one embodiment, the chemical cross-linking agent is 2-ethyl-2-(hydroxymethyl)-1,3-propanediol (also known as trimethylolpropane, as available from Sigma Aldrich Corp.), preferably from about 0 to 5 weight percent, more preferably from about 0.1 to 1 weight percent, and perhaps even more preferably from about 0.15 to 0.3 weight percent, based on the total weight of the polymer.

Additionally, and optionally, a polymer system of the present invention can contain at least one or more biocompatible catalysts that can assist in controlling the curing process, including the following periods: (1) the cure induction period, and (2) the full curing period of the biomaterial. Together these two periods, including their absolute and relative lengths, and the rate of acceleration or cure within each period, determine the cure kinetics or profile for the composition. In some embodiments, however, a catalyst is not included. For instance embodiments in which the biomaterial is heated in the course of curing, such as in a heated mold in the manner described herein, can performed without the use of a catalyst.

Some examples of suitable catalysts for preparation of the formed PU of this invention include, but are not limited to, tin and tertiary amine compounds or combinations thereof such as dibutyl tin dilaurate (DBTDL), and tin or mixed tin catalysts including those available under the tradenames “Cotin 222”, “Fomrez UL-22” (Crompton Corp.), “dabco” (a triethylene diamine from Sigma-Aldrich), stannous octanoate, trimethyl amine, and triethyl amine.

In one embodiment of the invention, the catalyst is bis-(dodecylthio)-dimethylstannane (available from Crompton Corp. as Fomrez catalyst UL-22), preferably from about 0 to 2 weight percent, more preferably from about 0 to 1 weight percent, and perhaps most preferably from 0.0009 to 0.002 weight percent, based on the total weight of the polymer.

Further, a polymer stabilizer additive useful for protecting the polymer from oxidation can be included. In one embodiment of the invention, the additive is pentaerythritol tetrakis (3-(3,5-di-tert-buyl-4-hydroxyphenyl)proprionate (available from Ciba Specialty Chemicals, Inc. as Irganox 1010), preferably from about 0 to 5 weight percent, more preferably about 0.1 to 1 weight percent, and perhaps even more preferably about 0.35 to 0.5 weight percent, based on the total weight of the polymer.

Optionally, other ingredients or additives can be included, for instance, a reactive polymer additive can be included from the group consisting of hydroxyl- or amine-terminated compounds selected from the group consisting of poybutadiene, polyisoprene, polyisobutylene, silicones, polyethylene-propylenediene, copolymers of butadiene with acryolnitrile, copolymers of butadiene with styrene, copolymers of isoprene with acrylonitrile, copolymers of isoprene with styrene, and mixtures of the above. Other additives can also be optionally provided. For example, catalysts such as Dabco, antioxidants such as vitamin E, hydrophobic additives such as hydroxyl-terminated polybutadiene, and dye green GLS, singularly or in combination, can be included in the polymer formulation.

The composition of the present invention provides a polyurethane that can be prepared ex vivo. Particularly when formed ex vivo, products incorporating the composition of this invention can be made in advance of their use, on a commercial scale, and under stringent conditions.

Polymeric biomaterials of this invention, including preferred polyurethanes can be prepared using automated manufacturing processes within the skill of those in the art. A preferred manufacturing method, for instance, includes the use of multichannel dispensing equipment to inject the polymer. Such equipment is well suited to high precision applications, having a variable or fixed number of channels, some have all channels dispensing the same volume while in others the volume can be set by channel, some have all channels dispensing the same fluid, while others allow for different fluids in different channels. The dispensing can be automated repetitive or manual. Suitable devices for metering, mixing and dispensing materials such as urethanes are commercially available from a variety of sources, including for instance from Adhesive Systems Technology Corp., 9000 Science Center Drive, New Hope, Minn. 55428.

Furthermore, polymeric biomaterials of this invention can be cured in a heated mold. The mold can receive the contents of the polymeric biomaterial before it is cured. In one embodiment, a permanent enclosed mold is used to form at least a part of the implant. Such a mold can be similar to a standard injection mold and have the ability to withstand large clamping forces. Further, such a mold can include runners and/or vents to allow material to enter and air to exit. Such a mold can be constructed from metals, polymers, ceramics, and/or other suitable materials. The mold can be capable of applying and controlling heat to the biomaterial to accelerate curing time. In some embodiments, the mold can be coated with a release coating agent to facilitate ease of removal of the cured biomaterial from the mold. Examples of suitable release agents include Teflon,™ silicone, florinated ethylene propylene (FEP), dichronite, gold, and nickel-Teflon combinations, various types of which are commercially available from a variety of sources, e.g., McLube Division of McGee Industries. In addition, the mold can be provided in two separable parts to further facilitate removal of the cured biomaterial.

Further, time and temperature parameters can be modified in processing to change the characteristics of the implant. A time temperature profile may be selected to achieve certain implant properties. In embodiments formed with a heated mold as described above, those skilled in the art will appreciate the manner in which both the temperature of the mold as well as the time biomaterial is maintained can be adjusted to change the characteristics of the molded implant.

In the embodiment in which an ex vivo curing polymer is used, the present invention preferably provides a biomaterial in the form of a curable polyurethane composition comprising a plurality of parts capable of being at least partially mixed at a time before use, the parts including: (1) a polymer component comprising the reaction product of one or more polyols, and one or more diisocyanates, and (2) a curative component comprising one or more chain extenders, one or more catalysts, and optionally, one or more polyols and/or other optional ingredients.

In some embodiments, long term congruence of the biomaterial is facilitated by its hydration in vivo, permitting the biomaterial to become more pliable, and in turn, facilitate congruence with the supporting tissue (e.g., bone). In turn, an increase in hydration and/or changes in temperature can improve the fit and mechanical lock between the implant and the supporting tissue. The biomaterial can be hydrated ex vivo and/or in vivo, both before and after the composition is cured. Preferably, the biomaterial can be further hydrated within the joint site after the composition in order to enhance both conformance and performance of the implant.

Implantable compositions of this invention demonstrate an optimal combination of properties, particularly in terms of their physical/mechanical properties, and biocompatibility. Such performance can be evaluated using procedures commonly accepted for the evaluation of natural tissue, as well as the evaluation of materials and polymers in general. In particular, a preferred composition, in its cured form, exhibits physical and mechanical properties that approximate or exceed those of the natural tissue it is intended to provide or replace. Fully cured polymeric (e.g., polyurethane) biomaterials within the scope of this invention provide an optimal combination of such properties as abrasion, compressive hardness, compressive modulus hardness, specific gravity, tear resistance, tensile strength, ultimate elongation, tensile modulus, and biocompatibility.

Physical/Mechanical Properties and Test Methods

The preferred property ranges given below are only relevant to certain embodiments of the invention. It will be appreciated by those reasonably skilled in the art that materials having one or more properties outside the scope of the preferred ranges given below are suitable for use with the present invention.

For example, in embodiments of the implant that have a first polymeric portion and a second polymeric portion having different properties, the second polymeric portion can comprise a material having a hardness of about 55 Shore D to about 75 Shore D (e.g., about 65 Shore D) and compression modulus of about 10,000 psi to about 13,000 psi (e.g., about 11,500 psi), and the first polymeric portion can comprise a material having a hardness of about 80 Shore A to about 90 Shore A (e.g., about 85 Shore A), and a compressive modulus of about 1,500 psi to about 4,500 psi (e.g., about 3,000 psi), as discussed further herein.

Abrasion values for a polymer can be determined with a rotating cylindrical drum device, known as a DIN abrader. A loaded cylindrical test piece is traversed along an abrasive cloth attached to a rotating drum, and the mass loss is measured after a specified length of travel. Advantages of this device include the use of a test piece small enough to be cut from a product or a comparatively thin sheet and a much reduced risk of abrasive contamination caused by debris or smearing. The result can be expressed with the abrasion resistance index, which is the ratio of the volume loss of a black standard rubber sample to the volume loss of the test sample.

The polymer preferably provides a DIN abrasion value of less than about 70 mm³, more preferably less than about 60 mm³ and most preferably less than about 50 mm³, as determined by AS™ Test Method D5963-96 (“Standard Test Method for Rubber Property Abrasion Resistance Rotary Drum Abrader”). DIN abrasion values of greater than about 70 mm³ tend to exhibit wear rates that are too great for longer term use as articulating surface.

The term hardness has been applied to scratch resistance and to rebound resilience, but for polymers it is taken to refer to a measure of resistance to indentation. The mode of deformation under an indentor is a mixture of tension, shear, and compression. The indenting force is usually applied in one of the following ways: Application of a constant force, the resultant indentation being measured, measurement of the force required to produce a constant indentation, or use of a spring resulting in variation of the indenting force with depth of indentation.

A biomaterial of this invention preferably provides a hardness value when hydrated of less than about 75 Shore D, and more preferably less than about 70 Shore D, as determined by AS™ Test Method D2240. In some embodiments, hydration of the biomaterial can lower the shore hardness value to less than about 60 Shore D to provide for greater congruency of the implant to the joint in situ.

To measure tensile modulus, tensile strength, and ultimate elongation, a test piece of the material is stretched until it breaks, and the force and elongation at various stages is measured. A tensile machine is used to perform this test. Generally, the basic elements of a tensile machine are grips to hold the test piece, a means of applying a strain (or stress), a force-measuring instrument, and an extensometer.

The polymer preferably provides a tensile modulus at 100% elongation value of about 1,000 psi to 10,000 psi, more preferably about 2,000 psi to 5,000 psi, and most preferably about 2,500 psi to 4,500 psi, as determined by AS™ Test method D412.

The polymer preferably provides a tensile modulus at 200% elongation value of about 1,000 psi to 10,000 psi, more preferably about 2,000 psi to 6,000 psi, and most preferably about 2,500 psi to 5,000 psi, as determined by AS™ Test method D412.

The polymer preferably provides a tensile strength value of greater than about 6,000 psi, more preferably greater than about 6,500 psi, and most preferably greater than about 7,000 psi., as determined by AS™ Test Method D412.

Preferably, the polymer provides an ultimate elongation of greater than about 200%, more preferably greater than about 250%, and most preferably greater than about 275%, as determined by AS™ Test Method D412.

To measure compressive modulus and compressive strength, a sample is formed in a standardized (e.g., puck) shape and varying compressive loads are applied to the sample in order to develop a corresponding curve. The compressive modulus can be determined from this curve. Compressive strength can be determined by applying increasing loads to a sample until the sample fails.

Preferably, the sample implant provides an compressive modulus of greater than about 4,000 psi, more preferably greater than about 4,500 psi, and most preferably greater than about 5,000 psi, as determined in the manner described above. In some embodiments, the compressive modulus can be greater than about 10,000 psi.

Preferably, the sample implant also provides a compressive strength of greater than about 6,000 psi, more preferably greater than about 7,000 psi, and most preferably greater than about 8,000 psi, as determined by a test similar to the one described above.

Although this invention has been described in detail with particular reference to preferred embodiments, it will be understood that it is intended to cover all modifications, variations and equivalents within the spirit and scope of this invention as described before.

What is claimed is:

1. An implant for an upper extremity joint arthroplasty, the implant comprising a first polymeric portion adapted to be retained within a bone of the upper extremity and a second polymeric portion having an articulating surface adapted to articulate against an opposing bone.

2. An implant according to claim 1, wherein the bone of the upper extremity is a radius and the opposing bone is selected from the group of an ulna, a humorous, and combinations thereof.

3. An implant according to claim 1, wherein the bone of the upper extremity is an ulna and the opposing bone is selected from the group of a radius, a carpal, and combinations thereof.

4. An implant according to claim 1, wherein the implant comprises one or more biomaterials selected from the group of polyurethane, polyethylene, poly(ether ether ketone), polyurethane functionalized polyethylene mixes, polyurethane siloxane mixtures, polystyrene, polyamides, silicones, acrylics and combinations thereof.

5. An implant according to claim 1, wherein the implant comprises polyurethane.

6. An implant according to claim 1, wherein the first polymeric portion comprises a shaft portion and the second polymeric portion comprises a head portion.

7. An implant according to claim 6, wherein the head portion comprises a relatively harder biomaterial than the shaft portion.

8. An implant according to claim 7, wherein the head portion comprises a polyurethane and the shaft portion comprises a relatively softer polyurethane.

9. An implant according to claim 8, wherein the two different polyurethanes are co-polymerized to form a continuous polyurethane material across a junction of the head portion and the shaft portion of the implant.

10. An implant according to claim 6, wherein both the head portion and the shaft portion comprise a material having a softer modulus than natural bone.

11. An implant according to claim 6, wherein the head portion and the shaft portion are joined at a junction.

12. An implant according to claim 6, wherein the shaft portion comprises a cylindrical shape with a rounded bullet shaped end adapted to ease insertion into the bone of the upper extremity.

13. An implant according to claim 6, wherein the head portion and the shaft portion each comprise a cylindrical shape, the diameter of the head portion being greater than the diameter of the shaft portion.

14. An implant according to claim 13, wherein the head portion comprises a cylindrical shape having radiused edges.

15. An implant for an upper extremity joint arthroplasty, the implant comprising a first polymeric portion adapted to be retained within a bone of the upper extremity and a second polymeric portion having an articulating surface adapted to articulate against an opposing bone, wherein the first polymeric portion comprises a shaft portion and the second polymeric portion comprises a head portion, and further wherein the implant comprises one or more biomaterials selected from the group of polyurethane, polyethylene, poly(ether ether ketone), polyurethane functionalized polyethylene mixes, polyurethane siloxane mixtures, polystyrene, polyamides, silicones, acrylics and combinations thereof.

16. An implant according to claim 15, wherein the bone of the upper extremity is a radius and the opposing bone is selected from the group of an ulna, a humorous, and combinations thereof.

17. An implant according to claim 15, wherein the bone of the upper extremity is an ulna and the opposing bone is selected from the group of a radius, a carpal, and combinations thereof.

18. An implant according to claim 15, wherein the implant comprises polyurethane.

19. An implant according to claim 15, wherein the head portion comprises a polyurethane and the shaft portion comprises a relatively softer polyurethane.

20. A method of placing an implant in an upper extremity joint, the method comprising providing an implant comprising a first polymeric portion and a second polymeric portion having an articulating surface, inserting the first polymeric portion within a bone of the upper extremity, and placing the articulating surface against an opposing bone.
